SEQUENCE LISTING (1 sequence)

SEQ ID NO: 1 - protein, 9 aa, Artificial sequence
  Synthetic construct
RAHYNIVTF

(19) United States
(12) Patent Application Publication
(10) Pub. No.: US 2023/0323296 A1
(43) Pub. Date: Oct. 12, 2023

(54) STEM CELL-LIKE MEMORY T CELLS AND USES THEREOF

(71) Applicant: GEORGETOWN UNIVERSITY, Washington, DC (US)

(72) Inventors: Samir N. KHLEIF, Washington, DC (US); Vivek VERMA, Bethesda, MD (US)

(73) Assignee: GEORGETOWN UNIVERSITY, Washington, DC (US)

(21) Appl. No.: 18/042,405

(22) PCT Filed: Aug. 20, 2021
(86) PCT No.: PCT/US21/46853

§ 371 Date: Feb. 21, 2023

Related U.S. Application Data
(60) Provisional application No. 63/068,672, filed on Aug. 21, 2020.

(51) Int. Cl. C12N 5/0783; A61P 37/02

(52) U.S. Cl. CPC C12N 5/0636; A61P 37/02; C12N 2501/15; C12N 2501/999; C12N 2501/2304; C12N 2501/2312; C12N 2501/24; C12N 2501/2302

(57) ABSTRACT

Provided herein are compositions comprising CD4⁺ stem cell like memory T (T_{SCM}) cells and their uses in the treatment of cancer, infection and autoimmune disorders.

### CROSS-REFERENCE TO RELATED APPLICATION

This application claims priority to U.S. Provisional Application No. 63/068,672, filed Aug. 21, 2020, which is hereby incorporated by reference in its entirety for all purposes.

### BACKGROUND

Immunotherapy has emerged as a promising treatment for diseases such as cancer. For example, the Food and Drug Administration (FDA) has approved antibodies targeting programmed death (PD)-1 and its ligand PD-L1 for the treatment of several types of cancers. The clinical responses to these therapies, however, have been restricted to certain subsets of patients, limiting their usefulness in a wide range of cancers and diseases.

### SUMMARY

Provided herein are new immunotherapies for the treatment of cancer, including methods for producing stem cell-like memory T (T_{SCM}) cells. The methods comprise contacting CD4⁺ T cells in vitro, ex vivo or in vivo with an effective amount of an MEK1/2 inhibitor to produce CD4⁺ T_{SCM} cells. Some methods further comprise expanding the CD4⁺ T_{SCM} cells in culture. Some methods further comprise expanding the CD4⁺ T_{SCM} cells in culture. In some methods, the CD4⁺ T_{SCM} cells have a CD62L⁺CD44⁻ naïve-like phenotype. In some methods, the CD4⁺ T_{SCM} cells have an increased level of Sca1 as compared to untreated CD4⁺ T cells.

Optionally, the methods further comprise differentiating the T_{SCM} cells into one or more types of cells that are of CD4⁺ specific T cell-lineage. In some methods, the one or more types of cells of CD4⁺ T specific cell-lineage are selected from the group consisting of regulatory T cells (Tregs), Th1, Th2 and Th17 cells. For example, the type of cell of CD4⁺ T specific cell-lineage is a Treg cell, wherein differentiating the T_{SCM} cells into Treg cells comprises contacting the T_{SCM} cells with IL-2 and TGFβ. Optionally, the type of cell of CD4⁺ T specific cell-lineage is a Th1 cell, wherein differentiating the T_{SCM} cells into Th1 cells comprises contacting the T_{SCM} cells with IL-2, IL-12, IFN-γ and αIL-4. When the type of cell of CD4⁺ T specific cell-lineage is Th2 cell, differentiating the T_{SCM} cells into Th2 cells comprises contacting the T_{SCM} cells with IL-2, IL-4, αIL-12 and αIFN-γ. Optionally, the type of cell of CD4⁺ T specific cell-lineage is Th17 cell, and differentiating the T_{SCM} cells into Th17 cells comprises contacting the T_{SCM} cells with TGFβ and IL-6.

In some methods, the CD4⁺ T cells are contacted with an effective amount of an MEK1/2 inhibitor to generate multipotent CD4⁺ T_{SCM} cells, and the multipotent CD4⁺ T_{SCM} cells are subsequently contacted with cell-lineage specific inducing conditions to generate one or more types of cells that are of CD4⁺ T specific cell-lineage. In some methods, the CD4⁺ T cells are concurrently contacted with an effective amount of an MEK1/2 inhibitor and cell-lineage specific inducing conditions to generate one or more types of T_{SCM} cells that are of CD4⁺ T specific cell-lineage.

Any MEK1/2 inhibitor can be used in the methods described herein. Without meaning to be limiting, the MEK1/2 inhibitor is optionally Selumetinib. In some methods, the CD4⁺ T cells are genetically engineered CD4⁺ T cells or generated by any other means. For example, the CD4⁺ T cells can be genetically engineered to express a chimeric antigen receptor.

Also provided is a method for treating an infection or cancer in a subject comprising contacting CD4⁺ T cells ex vivo with an effective amount of an MEK1/2 inhibitor to produce T_{SCM} cells and administering the T_{SCM} cells to the subject with an infection or cancer. In some methods, the T_{SCM} cells are expanded prior to administration to the subject. Optionally, the methods further comprise differentiating the T_{SCM} cells into Th1, Th2 or Th17 cells prior to administration to the subject. The CD4⁺ T cells can be genetically engineered CD4⁺ T cells, for example, genetically engineered to express a chimeric antigen receptor. The CD4⁺ T cells are derived from a suitable source and can be autologous (i.e., from the same subject that is administered the T_{SCM} cells), allogeneic (i.e., from a donor that is not the same subject), or heterologous (i.e., from a different species).

The methods for treating an infection or cancer in a subject can further comprise administering an effective amount of a second therapeutic agent to the subject. The second therapeutic agent is optionally selected from the group consisting of an immunomodulatory agent, a vaccine, a tumor antigen or a pathogen antigen. In some methods, the immunomodulatory agent is an antibody or an antigen binding fragment thereof that binds to PD1, PDL1, OX40, CTLA-4, TIM-3, TIGIT, VISTA, BTLA, LAG-3, CD27, KIR, A2AR or GITR. The immunomodulatory agent can be an immunosuppressant or an immunostimulant depending upon the desired action.

Also provided is a method for treating an autoimmune disorder in a subject comprising contacting CD4⁺ T cells ex vivo with an effective amount of an MEK1/2 inhibitor to produce T_{SCM} cells and administering the T_{SCM} cells to the subject with an autoimmune disorder. Optionally, the T_{SCM} cells are expanded prior to administration to the subject. Some methods further comprise differentiating the T_{SCM} cells into Treg cells prior to administration to the subject. The CD4⁺ T cells can be genetically engineered CD4⁺ T cells, for example, CD4⁺ T cells genetically engineered to express a chimeric antigen receptor. The CD4⁺ T cells can be autologous, allogeneic, or heterologous. Optionally, the methods for treating an autoimmune disorder further comprise administering an effective amount of an immunosuppressant to the subject.

Also provided is an in vivo method of increasing CD4⁺ T_{SCM} cells in a subject comprising administering an MEK1/2 inhibitor to the subject. The MEK1/2 inhibitor is optionally administered to the subject in combination with CD4⁺ T_{SCM} cells produced by any of the methods described herein.

Further provided is a pharmaceutical composition comprising a T_{SCM} cell or cell derived therefrom, wherein the T_{SCM} cell is produced by contacting CD4⁺ T cells in vitro or ex vivo with an effective amount of an MEK1/2 inhibitor. The composition optionally includes a second therapeutic agent, such as an immunomodulator, a vaccine, a tumor-specific antigen or a pathogen-specific antigen.

Also provided are methods of treating cancer, an infection or an autoimmune disorder comprising administering to a subject any of the compositions described herein.

### DESCRIPTION OF THE FIGURES

FIG. 1A shows that MEK1/2 inhibitor increases CD4⁺ T cells in the tumor micro environment. Mice were treated and tumors were isolated. The frequency of CD4⁺ cells in variously treated mice (UT, untreated; Vax; MEKi, MEK1/2 inhibitor only; and Vax +MEKi, both E7 and MEK inhibitor) were determined. Error bars represent mean±SEM. (^{NS}non-significant, *p≤0.05, **p≤0.01). For Vax treated mice, The CTL epitope from HPV16 E749-57 (RAHYNIVTF, 100 μg/mouse) was used mixed with PADRE, a small 13-mer non-natural pan HLA DR-binding sequence that is a potent T helper cell epitope (20 μg/mouse-Celtek Bioscience, Franklin, TN), and QuilA (adjuvant, 10 μg/mouse-Brenntag, Westbury, NY). Two doses of the vaccine were administered subcutaneously (s.c.) every seven days starting at an average tumor volume of 0.07-0.08 cm³. Three days after the second vaccination, mice were sacrificed and expression of various markers was analyzed in cells from the tumors by flow cytometry

FIG. 1B shows that MEK1/2 inhibitor induces CD4⁺ T cells with a naïve-like phenotype (CD62L⁺CD44⁻) in the tumor microenvironment. The frequency of CD62L⁺CD44⁻ CD4⁺ T cells in variously treated mice as described for FIG. 1A were determined. Error bars represent mean±SEM. (^{NS}non-significant, *p≤0.05, **p≤0.01).

FIG. 2A shows that MEK1/2 inhibitor increases induction of T_{SCM} in vitro. T conventional (T_{CONV}) cells (from mouse spleen), defined as CD4⁺FoxP3⁻ cells, were activated (Act) by IL-2 with anti-CD3/anti-CD28 in the presence (+) or absence (−) of MEK1/2 inhibitor for 72 hours, and the number of Sca1⁺CD62L⁺CD44⁻CD4⁺ T cells was determined by flow cytometry sorting (FACS).

FIG. 2B shows that MEK1/2 inhibitor-induced T_{SCM} cells have enhanced self-renewal capacity and multipotency index. FACS-sorted T_{SCM} and T_{CM} cells in MEK1/2 inhibitor-treated CD4 T cells (treated as in FIG. 2A) were re-challenged for another 72 hours followed by determination of a multipotency index by estimating the generation of T_{SCM}, T_{CM}, and TEM cells from each respective population. Error bars represent mean±SEM. (*p≤0.05, **p≤0.01, ***p≤0.001, ****p≤0.0001).

FIG. 3A shows that MEK1/2 inhibition generates CD4⁺ lineage-specific T_{SCM} cells in the presence of Th17-inducing conditions. FACS-sorted CD4⁺ T cells from spleens of WT mice were cultured for 72 hours in the presence of IL-2/anti-CD3/anti-CD28 (Th0 conditions) with MEK1/2 inhibitor (Th0⁺ MEKi) or without MEK1/2 inhibitor (Th0). Also, within the same experiment, naive CD4⁺ T cells were activated in Th17 lineage-specific conditions: ((Ind) TGFβ (2.5 ng/mL)+IL-6 (100 ng/mL)). After incubation, cells were stained for ROR-γ and analyzed by flow cytometry as a readout of lineage-specific cells on Sca1⁺CD62L⁺CD44⁺ cells. Error bars represent mean±SEM. (*p≤0.05, **p≤0.01, ***p≤0.001, ****p≤0.0001).

FIG. 3B shows that MEK1/2 inhibition generates CD4⁺ lineage-specific T_{SCM} cells in the presence of Treg-inducing conditions. FACS-sorted CD4⁺ T cells from spleens of WT mice were cultured for 72 hours in the presence of IL-2/anti-CD3/anti-CD28 (Th0 conditions) with MEK1/2 inhibitor (Th0⁺ MEKi) or without MEK1/2 inhibitor (Th0). Also, within the same experiment, naive CD4⁺ T cells were activated in Treg-specific conditions: (IL-2 (100 U/mL)⁺ (Ind) TGFβ (2.5 ng/mL)). After incubation, cells were stained for FoxP3 and analyzed by flow cytometry as a readout of lineage-specific cells on Sca1⁺CD62L⁺CD44⁺ cells. Error bars represent mean±SEM. (*p≤0.05, **p≤0.01, ***p≤0.001).

FIG. 3C shows that MEK1/2 inhibition generates CD4⁺ lineage-specific T_{SCM} cells in the presence of Th1-inducing conditions. FACS-sorted CD4⁺ T cells from spleens of WT mice were cultured for 72 hours in the presence of IL-2/anti-CD3/anti-CD28 (Th0 conditions) with MEK1/2 inhibitor (Th0⁺ MEKi) or without MEK1/2 inhibitor (Th0). Also, within the same experiment, naive CD4⁺ T cells were activated in Th1-specific conditions: (IL-2 (100 U/mL)⁺ IL-12 (10 ng/mL)⁺ IFN-γ (10 ng/mL)+αIL-4 (10 μg/mL)). After incubation, cells were stained for Tbet and analyzed by flow cytometry as a readout of lineage-specific cells on Sca1⁺CD62L⁺CD44⁺ cells. Error bars represent mean±SEM. (*p≤0.05, **p≤0.01).

FIG. 3D shows that MEK1/2 inhibition generates CD4⁺ lineage-specific T_{SCM} cells in the presence of Th2-inducing conditions. FACS-sorted CD4⁺ T cells from spleens of WT mice were cultured for 72 hours in the presence of IL-2/anti-CD3/anti-CD28 (Th0 conditions) with MEK1/2 inhibitor (Th0⁺MEKi) or without MEK1/2 inhibitor (Th0). Also, within the same experiment, naive CD4⁺ T cells were activated in Th2-specific conditions: (IL-2 (100 U/mL)⁺ IL-4 (30 ng/mL)+αIL-12 (10 μg/mL)+αIFN-γ (10 μg/mL)). After incubation, cells were stained for GATA and analyzed by flow cytometry as a readout of lineage-specific cells on Sca1⁺CD62L⁺CD44⁺ cells. Error bars represent mean±SEM. (*p≤0.05, **p≤0.01).

FIG. 4A shows that MEK1/2 inhibitor-induced T_{SCM} cells are multipotent as they generate Th17 cells in the presence of cell skewing conditions. FACS-sorted CD4⁺ T cells from spleens of wild-type mice were cultured for 72 hours in the presence of IL-2/anti-CD3/anti-CD28 (Th0 condition) with or without MEK1/2 inhibitor. After 72 hours, T_{SCM} (Sca1⁺CD62L⁺CD44⁻) and T_{CM} (CD62L⁺CD44⁺) cells were FACS-sorted and further activated with IL-2/anti-CD3/anti-CD28 (Th0) or Th17 cell skewing conditions for 24 hours (TGFβ (2.5 ng/mL)+IL-6 (100 ng/mL)). After incubation, cells were stained for ROR-γ and analyzed by flow cytometry as a readout of lineage-specific CD4⁺ cells. Error bars represent mean±SEM. (*p≤0.05, **p≤0.01, ***p≤0.001, ****p≤0.0001).

FIG. 4B shows that MEK1/2 inhibitor-induced T_{SCM} cells are multipotent as they generate Treg cells in the presence of cell skewing conditions. FACS-sorted CD4⁺ T cells from spleens of wild-type mice were cultured for 72 hours in the presence of IL-2/anti-CD3/anti-CD28 (Th0 condition) with or without MEK1/2 inhibitor. After 72 hours, T_{SCM} (Sca1⁺CD62L⁺CD44⁻) and T_{CM} (CD62L⁺CD44⁺) cells were FACS-sorted and further activated with IL-2/anti-CD3/anti-CD28 (Th0) or Treg cell skewing conditions for 24 hours (IL-2 (100 U/mL)+TGFβ (2.5 ng/mL)). After incubation, cells were stained for FoxP3 and analyzed by flow cytometry as a readout of lineage-specific CD4⁺ cells. Error bars represent mean±SEM. (*p≤0.05, **p≤0.01, ***p≤0.001, ****p≤0.0001).

FIG. 4C shows that MEK1/2 inhibitor-induced T_{SCM} cells are multipotent as they generate Th1 cells in the presence of cell skewing conditions. FACS-sorted CD4⁺ T cells from spleens of wild-type mice were cultured for 72 hours in the presence of IL-2/anti-CD3/anti-CD28 (Th0 condition) with or without MEK1/2 inhibitor. After 72 hours, T_{SCM} (Sca1⁺CD62L⁺CD44⁻) and T_{CM} (CD62L⁺CD44⁺) cells were FACS-sorted and further activated with IL-2/anti-CD3/anti-CD28 (Th0) or Th1 cell skewing conditions for 24 hours (IL-2 (100 U/mL)+IL-12 (10 ng/mL)+IFN-γ (10 ng/mL)+αIL-4 (10 μg/mL)). After incubation, cells were stained for Tbet and analyzed by flow cytometry as a readout of lineage-specific CD4⁺ cells. Error bars represent mean±SEM. (*p≤0.05, **p≤0.01, ***p≤0.001, ****p≤0.0001).

FIG. 4D shows that MEK1/2 inhibitor-induced T_{SCM} cells are multipotent as they generate Th2 cells in the presence of cell skewing conditions. FACS-sorted CD4⁺ T cells from spleens of wild-type mice were cultured for 72 hours in the presence of IL-2/anti-CD3/anti-CD28 (Th0 condition) with or without MEK1/2 inhibitor. After 72 hours, T_{SCM} (Sca1⁺CD62L⁺CD44⁻) and T_{CM} (CD62L⁺CD44⁺) cells were FACS-sorted and further activated with IL-2/anti-CD3/anti-CD28 (Th0) or Th2 cell skewing conditions for 24 hours (IL-2 (100 U/mL)+IL-4 (30 ng/mL)+αIL-12 (10 μg/mL)+αIFN-γ (10 μg/mL)). After incubation, cells were stained for GATA and analyzed by flow cytometry as a readout of lineage-specific CD4⁺ cells. Error bars represent mean±SEM. (*p≤0.05, **p≤0.01, ***p≤0.001, ****p≤0.0001).

FIG. 5 is a schematic showing that MEK1/2 inhibition induces CD4⁺ T_{SCM} cells that have self-renewal capacity with potent recall response (Result 1), showing that cell lineage-specific CD4⁺ T_{SCM} cells can be generated by providing specific cell skewing conditions (Result 2), and showing that the CD4⁺ T_{SCM} cells generated after MEK1/2 inhibition are multipotent (i.e., capable of generating other CD4 T cell phenotypes after their TCR-mediated activation (Result 3).

FIG. 6A is a schematic showing an exemplary timeline for anti-tumor treatment with an MEK1/MEK2 inhibitor and an anti-OX40 antibody.

FIG. 6B shows that treatment with an MEK inhibitor (6244), in combination with an anti-OX40 antibody, significantly reduced tumor growth.

FIG. 6C shows that treatment with an MEK inhibitor (6244), in combination with an anti-OX40 antibody, led to a significant increase in mice survival.

### DETAILED DESCRIPTION

CD4⁺ T cells constitute an important determinant of anti-tumor outcome for many treatments. Since CD4⁺ T cells recognize antigen in the context of MHC class II found primarily on immune cells, their key role is to modulate the state and function of other immune cells (Ahrends and Borst “The opposing roles of CD4(⁺) T cells in anti-tumour immunity,” Immunology, 154(4):582-592 (2018); Ostroumov et al. “CD4 and CD8 T lymphocyte interplay in controlling tumor growth. Cell Mol Life Sci 75: 689-713 (2018)). Moreover, CD4⁺ T cells represent a diverse cell population with many differentiation states that have all been shown to control immune responses against cancer (Kennedy and Celis “Multiple roles for CD4⁺ T cells in anti-tumor immune responses. Immunol Rev 222: 129-144 (2008)). However, the effects of MEK inhibition on the CD4⁺ T cells were largely unknown. The role of the MEK pathway in regulating the generation of CD4⁺ T_{SCM} and the ability of these T cells to differentiate into specialized CD4 lineages are described herein. Specifically, MEK1/2 inhibition induces CD4⁺ T_{SCM} cells that have self-renewal capacity with potent recall response. T_{SCM} cells of CD4⁺ T cell specific lineage can be generated by providing specific cell skewing conditions. Further, the CD4⁺ T_{SCM} generated after MEK1/2 inhibition are multipotent (i.e., these T_{SCM} cells are able to generate other CD4⁺ T cell phenotypes after their T cell receptor (TCR)-mediated activation). As used throughout the terms multipotent and pluripotent are used interchangeable. The ability to re-engineer CD4⁺ cells into T_{SCM} is a novel approach for generation of superior therapeutic T cells that last longer and can treat a variety of conditions.

### Methods of Producing T_{SCM} Cells

Provided herein are methods for inducing CD4⁺ T cells to have a T_{SCM} phenotype. The methods comprise contacting CD4⁺ T cells in vitro or ex vivo with an effective amount of an MEK1/2 inhibitor to produce T_{SCM} cells, i.e., CD4⁺ T_{SCM} cells.

As used herein, a T cell or T lymphocyte refers to a lymphoid cell that expresses a T cell receptor molecule. CD4⁺ T cells are T lymphocytes that, after being activated and differentiated into distinct effector subtypes (for example, Th1, Th2, Th17 or Treg cells), play a major role in mediating immune responses through the secretion of specific cytokines.

CD4⁺ T cells carry out multiple functions, including activating immune cells and playing a critical role in the suppression of immune reactions. As used herein, an immune cell refers to any cell of hematopoietic origin including, but not limited to, T cells, B cells, monocytes, dendritic cells and macrophages.

In some methods, the CD4⁺ T cell contacted with an MEK1/2 inhibitor is a primary T cell. As used herein, a primary T cell is a T cell that has not been previously transformed or immortalized. Such primary cells can be cultured, sub-cultured, or passaged a limited number of times (e.g., cultured 0, 1, 2, 3, 4, 5, 6, 7, 8, 9, 10, 11, 12, 13, 14, 15, 16, 17, 18, 19, or 20 times). In some cases, the primary cells are adapted to in vitro culture conditions. In some cases, the primary cells are isolated from an organism, system, organ, or tissue; optionally sorted; and utilized directly without culturing or sub-culturing.

In some cases, CD4⁺ T cells, for example primary CD4⁺ T cells, are stimulated, activated, or differentiated. For example, CD4⁺ T cells can be activated by contact with (e.g., culturing in the presence of) an anti-CD3 antibody, an anti-CD28 antibody, IL-2, IFN-γ, IL-7, or a combination thereof.

As used throughout, T_{SCM} cells are a subset of memory lymphocytes that possess a stem cell-like ability to self-renew and the multipotent capacity to reconstitute the entire spectrum of memory and effector subsets. Functionally, T-cells having the T_{SCM} phenotype have been shown to have enhanced anti-tumor responses compared to both naive and memory T cells (Golubovskaya, V. and Wu, L., Cancers, 8(3): 36 (2016)), which seem to depend upon their long-term persistence, self-renewability and ability to differentiate into effector T-cells (TEFF) (Graef, P., et al., Immunity, 41:116-126 (2014)).

In the ex vivo or in vitro methods provided herein, the MEK1/2 inhibitor can be added to CD4⁺ T cells, in culture, until the CD4⁺ T cells develop a T_{SCM} phenotype, i.e., a CD62L⁺CD44⁻naïve-like phenotype that also express stem cell antigen 1 (Sca1) (i.e., a Sca1+CD62L+CD44− phenothpe), and can have lower mitochondrial lower mitochondrial membrane potential, for example, as measured by tetramethylrhodamine methyl ester (TMRM dye). Hence, these T_{SCM} cells can be identified as CD62L⁺CD44⁻Sca1⁺ TMRM^{low} CD4 T cells. The human counterpart of these T_{SCM} cells can be identified as CD45RA⁺CCR7+CD95⁺ TMRM^{low} CD4 cells. CD4⁺ T_{SCM} cells are also characterized by having an increased level of Sca1 as compared to untreated CD4⁺ T cells.

In the methods provided herein, the MEK1/2 inhibitor can be selected from the group consisting of TAK-733, Selumetinib, PD98059, Trametinib, PD184352, Rafametinib, U0126-EtOH and SL327. Other inhibitors include, but are not limited to a chemical, a small or large molecule (organic or inorganic), a protein, a peptide, a cDNA, an antibody, a morpholino, a triple helix molecule, an siRNA, a shRNA, an miRNA, an antisense RNA or a ribozyme that inhibits at least one activity of MEK1/2. Optionally, the CD4⁺ T cells are contacted with one or more MEK1/2 inhibitors.

The methods can further comprise expanding the CD4⁺ T_{SCM} cells in culture, for example, via antigen-stimulation. The methods can further comprise differentiating the CD4⁺ T_{SCM} cells into one or more types of cell-lineage specific CD4⁺ T cells. The one or more types of cell-lineage specific CD4⁺ T cells can be selected from the group consisting of T-regulatory (Treg), T-helper 1 (Th1), T-helper 2 (Th2) and T-helper 17 (Th17) cells. When the type of cell-lineage specific CD4⁺ T cells is Treg cell, for example, differentiating the T_{SCM} cells into Treg cells can comprise contacting the T_{SCM} cells with interleukin-2 (IL-2) and transforming growth factor beta (TGFβ). When the type of cell-lineage specific CD4⁺ T cell is Th1 cell, differentiating the T_{SCM} cells into Th1 cells can comprise contacting the T_{SCM} cells with IL-2, interleukin 12 (IL-12), interferon gamma (IFN-γ) and alpha interleukin 4 (αIL-4). When the type of cell-lineage specific CD4⁺ T cells is Th2 cell, differentiating the T_{SCM} cells into Th2 cells can comprise contacting the T_{SCM} cells with IL-2, interleukin 4 (IL-4), alpha interleukin 12 (αIL-12) and αIFN-γ. When the type of cell-lineage specific CD4⁺ T cells is Th17 cell, differentiating the T_{SCM} cells into Th17 cells can comprise contacting the T_{SCM} cells with TGFβ and interleukin 6 (IL-6).

In some methods, the CD4⁺ T cells are contacted with an effective amount of an MEK1/2 inhibitor to generate multipotent CD4⁺ T_{SCM} cells, and the multipotent CD4⁺ T_{SCM} cells are subsequently contacted with cell-lineage specific inducing conditions to generate one or more types of cells that are of CD4⁺ T specific cell-lineage.

In some methods, the CD4⁺ T cells are concurrently contacted with an effective amount of an MEK1/2 inhibitor and cell-lineage specific inducing conditions to generate one or more types of T_{SCM} cells that are of CD4⁺ T specific cell-lineage. CD4⁺ T cell subtypes can subsequently be generated from the cell-lineage specific CD4⁺ T_{SCM} cells.

The CD4⁺ T cells contacted with the MEK inhibitor(s) are optionally genetically engineered CD4⁺ T cells. For example, the CD4⁺ T cells can be genetically engineered to express a binding moiety that binds to a target protein or peptide. The target protein is optionally a cell surface protein, for example, a tumor specific antigen. Examples of tumor specific antigens include, but are not limited to, HER1, HER2, prostate surface antigen (PSA), human chorionic gonadotropin (HCG), glycosyltransferase-1,4-N-acetylgalactosaminyltransferases (GalNAc), NUC18, melanoma antigen gp75, human cytokeratin 8; high molecular weight melanoma antigen, keratin 19, MAGEA (CT1), BAGE (CT2), MAGEB (CT3), GAGE (CT4), SSX (CT5), NY-ESO-1 (CT6), MAGEC (CT7), SYCP1 (C8), SPANXB 1 (CT11.2), NA88 (CT18), CTAGE (CT21), SP A17 (CT22), OYTES-1 (CT23), CAGE (CT26), HOM-TES-85 (CT28), HCA661 (CT30), NY-SAR-35 (CT38), FATE (CT43), TPTE (CT44), α-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferase AS fusion protein, HLA-A2, HLA-All, hsp70-2, KIAA0205, Mart2, Mum-1, 2, and 3, neo-PAP, myosin class I, OS-9, pmlRARa fusion protein, PTPRK, K-ras, N-ras, Triosephosphate isomerase, Bage-1, Gage 3,4,5,6,7, GnTV, Herv-K-mel, Lage-1, Mage-S A1,2,3,4,6,10,12, Mage-C2, NA-88, NY-Eso-1/Lage-2, SP17, SSX-2, and TRP2-Int2, MelanA (MART-I), gpl00 (Pmell7), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, catenin, CDK4, Mum-1, p16, TAGE, PSMA, PSCA, CT7, telomerase, 43-9F, 5T4, 791Tgp72, a-fetoprotein, 13HCG, BCA225, BTAA, CA 125, CA 15-3 (CA 27.29\BCAA), CA 195, CA 242, CA-50, CAM43, CD68\KP1, C0-029, FGF-5, G250, Ga733 (EpCAM), HTgp-175, M344, MA-50, MG7-Ag, MOV18, NB\70K, NY-C0-1, RCAS1, SDCCAG16, TA-90 (Mac-2 binding protein\cyclophilin C-associated protein), TAAL6, TAG72, TLP, and TPS.

In certain methods, the CD4⁺ T cells are genetically engineered to express a chimeric antigen receptor. See for example, Newick et al. “Chimeric antigen receptor T-cell therapy for solid tumors,” Mol. Ther. Oncolytics 3: 16006 (2016); and Miliotou and Papadopoulou “Car T-cell Therapy: A New Era in Cancer Immunotherapy,” Curr. Pharm. Biotechnol. 19(i): 5-18 (2018).

Also provided is an in vivo method of increasing CD4⁺ T_{SCM} cells in a subject comprising administering an MEK1/2 inhibitor to the subject. The MEK1/2 inhibitor is optionally administered to the subject in combination with CD4⁺ T_{SCM} cells produced by any of the methods described herein.

### Methods of Treatment

Also provided is a method for treating an infection or cancer in a subject comprising contacting CD4⁺ T cells ex vivo with an effective amount of an MEK1/2 inhibitor to produce T_{SCM} cells and administering the T_{SCM} cells to the subject with an infection or cancer. In some methods, the T_{SCM} cells are expanded prior to administration to the subject. The methods can further comprise differentiating the T_{SCM} cells into Th1, Th2 or Th17 cells as described above prior to administration to the subject. The contacted CD4⁺ T cells are optionally recombinant or genetically engineered CD4⁺ T cells as described above to express, for example, a chimeric antigen receptor or a binding moiety that binds a target protein.

In some methods for treating cancer, CD4⁺ cells can be obtained by culturing a tumor biopsy from the subject in the presence of IL-2 to stimulate the growth of T cells that specifically target and kill the tumor cells. The tumor specific T cells can be harvested from culture and purified if necessary. The harvested T cells can be expanded in cell culture prior to administration to the subject. Additionally, the tumor specific T cells can be genetically modified to express a chimeric antigen receptor or a binding moiety that binds a target protein.

As used herein, cancer is a disease characterized by the rapid and uncontrolled growth of aberrant cells. Cancer cells can spread locally or through the bloodstream and lymphatic system to other parts of the body. The cancer can be a solid tumor. In some embodiments, the cancer is a blood or hematological cancer, such as a leukemia (e.g., acute leukemia; acute lymphocytic leukemia; acute myelocytic leukemias, such as myeloblastic, promyelocytic, myelomonocytic, monocytic, erythroleukemia leukemias and myelodysplastic syndrome; chronic myelocytic (granulocytic) leukemia; chronic lymphocytic leukemia; hairy cell leukemia), polycythemia vera, or lymphomas (e.g., Hodgkin's disease or non-Hodgkin's disease lymphomas (e.g., diffuse anaplastic lymphoma kinase (ALK) negative, large B-cell lymphoma (DLBCL); diffuse anaplastic lymphoma kinase (ALK) positive, large B-cell lymphoma (DLBCL); anaplastic lymphoma kinase (ALK) positive, ALK⁺anaplastic large-cell lymphoma (ALCL), acute myeloid lymphoma (AML))), multiple myelomas (e.g., smoldering multiple myeloma, non-secretory myeloma, osteosclerotic myeloma, plasma cell leukemia, solitary plasmacytoma and extramedullary plasmacytoma), Waldenstrom's macroglobulinemia, monoclonal gammopathy of undetermined significance, benign monoclonal gammopathy and heavy chain disease. Solid tumors include, by way of example, bone and connective tissue sarcomas (e.g., bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma), brain tumors (e.g., glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma), breast cancer (e.g., adenocarcinoma, lobular (small cell) carcinoma, intraductal carcinoma, medullary breast cancer, mucinous breast cancer, tubular breast cancer, papillary breast cancer, Paget's disease, and inflammatory breast cancer), adrenal cancer (e.g., pheochromocytoma and adrenocortical carcinoma), thyroid cancer (e.g., papillary or follicular thyroid cancer, medullary thyroid cancer and anaplastic thyroid cancer), pancreatic cancer (e.g., insulinoma, gastrinoma, glucagonoma, vipoma, somatostatin-secreting tumor, and carcinoid or islet cell tumor), pituitary cancers (e.g., Cushing's disease, prolactin-secreting tumor, acromegaly, and diabetes insipidus), eye cancers (e.g., ocular melanoma such as iris melanoma, choroidal melanoma, and ciliary body melanoma, and retinoblastoma), vaginal cancers (e.g., squamous cell carcinoma, adenocarcinoma, and melanoma), vulvar cancer (e.g., squamous cell carcinoma, melanoma, adenocarcinoma, basal cell carcinoma, sarcoma, and Paget's disease), cervical cancers (e.g., squamous cell carcinoma and adenocarcinoma), uterine cancers (e.g., endometrial carcinoma and uterine sarcoma), ovarian cancers (e.g., ovarian epithelial carcinoma, borderline tumor, germ cell tumor, and stromal tumor), esophageal cancers (e.g., squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma), stomach cancers (e.g., adenocarcinoma, fungating (polypoid), ulcerating, superficial spreading, diffusely spreading, malignant lymphoma, liposarcoma, fibrosarcoma, and carcinosarcoma), colon cancers, rectal cancers, liver cancers (e.g., hepatocellular carcinoma and hepatoblastoma), gallbladder cancers (e.g., adenocarcinoma), cholangiocarcinomas (papillary, nodular, and diffuse), lung cancers (e.g., non-small cell lung cancer, squamous cell carcinoma (epidermoid carcinoma), adenocarcinoma, large-cell carcinoma and small-cell lung cancer), testicular cancers (e.g., germinal tumor, seminoma, anaplastic, classic (typical), spermatocytic, nonseminoma, embryonal carcinoma, teratoma carcinoma, choriocarcinoma (yolk-sac tumor)), prostate cancers (e.g., adenocarcinoma, leiomyosarcoma, and rhabdomyosarcoma), penile cancers, oral cancers (e.g., squamous cell carcinoma), basal cancers, salivary gland cancers (e.g., adenocarcinoma, mucoepidermoid carcinoma, and adenoidcystic carcinoma), esopharyngeal cancers (e.g., squamous cell cancer and verrucous cancer), skin cancers (e.g., basal cell carcinoma, squamous cell carcinoma and melanoma, superficial spreading melanoma, nodular melanoma, lentigo malignant melanoma, acral lentiginous melanoma), kidney cancers (e.g., renal cell cancer, adenocarcinoma, hypernephroma, fibrosarcoma, transitional cell cancer (renal pelvis and/or ureter), Wilms' tumor), bladder cancers (e.g., transitional cell carcinoma, squamous cell cancer, adenocarcinoma, and carcinosarcoma). In addition, cancers include myxosarcoma, osteogenic sarcoma, endotheliosarcoma, lymphangio endothelio sarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas.

In the methods provided herein, an infection to be treated can be caused by a bacterium, virus, protozoan, helminth, fungal pathogens, parasitic pathogens or other microbial pathogens.

The infection or disease can be acute or chronic. An acute infection is typically an infection of short duration, while a chronic infection is a type of persistent infection that is eventually cleared. The infection can be caused by, for example, Actinomyces, Anabaena, Aspergillus, Bacillus, Bacteroides, Bdellovibrio, Bordetella, Borrelia, Campylobacter, Caulobacter, Chlamydia, Chlorobium, Chromatium, Clostridium, Coccidioides, Corynebacterium, Cytophaga, Deinococcus, Entamoeba, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, Yersinia, Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Trypanosoma cruzi, Entamoeba histolytica, Toxoplasma gondii, Trichomonas vaginalis and Schistosoma mansoni.

In some embodiments, the disclosed compositions are used to treat chronic infections, for example, infections in which T cell exhaustion or T cell anergy has occurred causing the infection to remain with the host over a prolonged period of time. Exemplary infections to be treated are chronic infections caused by a hepatitis virus, a human immunodeficiency virus (HIV), a human T-lymphotrophic virus (HTLV), a herpes virus, an Epstein-Barr virus, or a human papilloma virus.

Because viral infections are cleared primarily by T cells, an increase in T-cell activity is therapeutically useful in situations where more rapid or thorough clearance of an infective viral agent is beneficial to an animal or human subject. Thus, the CD4⁺ T_{SCM} cells described herein or cells differentiated therefrom can be administered for the treatment of local or systemic viral infections, including, but not limited to, infections associated with immunodeficiency (e.g., HIV), papilloma (e.g., HPV), herpes (e.g., HSV), encephalitis, influenza (e.g., human influenza virus A), and common cold (e.g., human rhinovirus) and other viral infections, caused by, for example, HTLV, hepatitis virus, respiratory syncytial virus, vaccinia virus, coronavirus (for example, SARS-CoV-2 (COVID-19) and rabies virus. The CD4⁺ T_{SCM} cells or cells differentiated therefrom can also be administered to treat viral skin diseases such as herpes lesions or shingles, or genital warts.

Also provided is a method for treating an autoimmune disorder in a subject comprising contacting CD4⁺ T cells ex vivo with an effective amount of an MEK1/2 inhibitor to produce T_{SCM} cells and administering the T_{SCM} cells to the subject with an autoimmune disorder. In some methods, the T_{SCM} cells are expanded prior to administration to the subject. Optionally, the methods further comprise differentiating the T_{SCM} cells into Treg cells prior to administration to the subject.

As used herein, an autoimmune disease is a disease where the immune system cannot differentiate between a subject's own cells and foreign cells, thus causing the immune system to mistakenly attack healthy cells in the body. Exemplary autoimmune diseases include, but are not limited to, inflammatory bowel disease, systemic lupus erythematosus, vasculitis, rheumatoid arthritis, Type 1 diabetes mellitus, myasthenia gravis, multiple sclerosis, psoriasis, Graves' disease, Hashimoto's thyroiditis, Sjögrens syndrome, and scleroderma.

In any of the methods of treatment, the CD4⁺ T cells can be autologous or autogeneic CD4⁺ T cells (i.e., from the same subject that receives the CD4⁺ T_{SCM} cells); homologous or allogeneic (i.e., from a donor subject of the same species); or heterologous (i.e., from a different species). For allogeneic cells, CD4⁺ T cells can be isolated from a donor subject by obtaining a peripheral blood cell composition from the donor, depleting the peripheral blood cell composition of CD8⁺ T cells, natural killer cells etc., and optionally expanding CD4⁺ T cells specific to an antigen, for example, a tumor antigen, by culturing the CD4⁺ T cells with the antigen. Optionally, the CD4⁺ T cell donor is HLA-matched, partially HLA-matched, or haploidentical to the recipient. In some methods the CD4⁺ T cells obtained from a subject can be cryopreserved prior to contacting the cells with an MEK 1/2 inhibitor. Optionally, the CD4⁺ T_{SCM} cells produced using any of the in vitro or ex vivo methods described herein are cryopreserved prior to expansion and/or administration to the subject.

Also provided is a method for treating an infection, cancer or an autoimmune disorder in a subject by administering an effective amount of an MEK1/2 inhibitor to the subject with an infection, cancer or an autoimmune disorder, thereby increasing T_{SCM} cells in the subject.

Optionally, the MEK1/2 inhibitor is administered to the subject in combination with CD4⁺ T_{SCM} cells.

Any of the treatment methods described herein can further comprise administering an effective amount of a second therapeutic agent to the subject. The second therapeutic agent can be selected from the group consisting of a chemotherapeutic agent, an adjuvant, an immunomodulatory agent, a vaccine, a potentiating agent, a tumor antigen, a pathogen antigen or a combination thereof. It is understood that combinations, for example, a composition comprising CD4⁺ T_{SCM} cells and a chemotherapeutic agent, can be administered either concomitantly (e.g., as an admixture), separately but simultaneously (e.g., via separate intravenous lines into the same subject), or sequentially (e.g., one of the compounds or agents is given first followed by the second). Any of the methods provided herein can further comprise radiation therapy or surgery.

As used herein modulate or modulation relates to altering an effect, result, or activity (e.g., signal transduction). Such modulation can be agonistic or antagonistic. Antagonistic modulation can be partial (i.e., attenuating, but not abolishing) or it can completely abolish such activity (e.g., neutralizing). Modulation can include internalization of a receptor following binding of an antibody or a reduction in expression of a receptor on the target cell. Agonistic modulation can enhance or otherwise increase or enhance an activity (e.g., signal transduction). In another example, modulation can alter the nature of the interaction between a ligand and its cognate receptor so as to alter the nature of the elicited signal transduction. In some cases, a non-cellular therapeutic agent described herein, for example, an antibody, can bind to a ligand or receptor, and alter its ability to bind to other ligands or receptors. In some embodiments, such modulation will provide at least a 10%, 20%, 30%, 40%, or 50% change in a measurable immune response, or at least a 2-fold, 5-fold, 10-fold, or at least a 100-fold change in a measurable immune response.

As used herein, an immune response is the development of a beneficial humoral (antibody mediated) and/or a cellular (mediated by antigen-specific T cells or their secretion products) response directed against a peptide in a recipient patient. Such a response can be an active response induced by administration of an immunogen or a passive response induced by administration of antibody or primed T-cells. A cellular immune response is elicited by the presentation of polypeptide epitopes in association with Class I or Class II molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils, activation or recruitment of neutrophils or other components of innate immunity. The presence of a cell-mediated immunological response can be determined by proliferation assays (CD4⁺ T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

Representative chemotherapeutic agents include, but are not limited to amsacrine, bleomycin, busulfan, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epirubicin, etoposide, fludarabine, fluorouracil, gemcitabine, hydroxycarbamide, idarubicin, ifosfamide, irinotecan, leucovorin, liposomal doxorubicin, liposomal daunorubicin, lomustine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, tegafur-uracil, temozolomide, teniposide, thiotepa, tioguanine, topotecan, treosulfan, vinblastine, vincristine, vindesine, vinorelbine, or a combination thereof. Representative pro-apoptotic agents include, but are not limited to fludarabinetaurosporine, cycloheximide, actinomycin D, lactosylceramide, 15d-PGJ(2) and combinations thereof.

As used herein, a potentiating agent acts to increase the immune response effected by the CD4⁺ T_{SCM} cells. Representative potentiating agents include, but are not limited to, cyclophosphamide (CTX, Cytoxan® (Baxter Healthcare, Deerfield, Illinois) or Neosar® (Teva, Petah Tikva, Israel) ifosfamide (IFO, Ifex), perfosfamide, trophosphamide (trofosfamide; Ixoten), and pharmaceutically acceptable salts, solvates, prodrugs and metabolites thereof (US Patent Application Publication No. 20070202077. Additional cyclophosphamide analogs are described in U.S. Pat. No. 5,190,929. Other potentiating agents include mafosfamide (NSC 345842), glufosfamide (D19575, j-D-glucosylisophosphoramide mustard), S-(−)-bromofosfamide (CBM-11), NSC 612567 (aldophosphamide perhydrothiazine) and NSC 613060 (aldophosphamide thiazolidine).

In some methods, the immunomodulator is an immunostimulant. As used herein an immunostimulant is an agent that stimulates or activates an immune response. Stimulating or activating an immune response includes inhibiting a suppressive immune response. Examples of immunostimulants include, but are not limited to antibodies that activate CD27, CD40, OX40, GITR, CD137, CD28, 4-1BB or ICOS signal transduction. Vaccines can also be used to stimulate an immune response.

In some methods, the immunomodulator is an immunosuppressant. As used herein, an immunosuppressant is an agent that suppresses or inhibits an immune response. Examples of immunosuppresants include, but are not limited to, calcineurin inhibitors (e.g., cyclosporin, tacrolimus), corticosteroids (e.g., methylprednisolone, dexamethasone, prednisolone) and cytotoxic immunosuppressants (e.g., azathioprine, chlorambucil, cyclophosphamide, mercaptopurine, methotrexate).

In some methods, the immunomodulator is an antibody or an antigen binding fragment thereof that binds to PD1, PDL1, OX40, CTLA-4, TIM-3, TIGIT, VISTA, BTLA, LAG-3, CD27, KIR, A2AR or GITR.

As used herein, the term antibody encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (κ) and lambda (Q), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG and IgM. Several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called alpha, delta, epsilon, gamma, and mu, respectively. The term variable is used herein to describe certain portions of the antibody domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework region (FR). The variable domains of native heavy and light chains each comprise four FR regions, largely adopting a R-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies. The constant domains are not involved directly in binding an antibody to an antigen but exhibit various effector functions, such as participation of the antibody in antibody-dependent cellular toxicity.

As used herein, an antigen binding fragment of an antibody refers to one or more portions of an antibody that contain the antibody's Complementarity Determining Regions (CDRs) and optionally the framework residues that include the antibody's variable region antigen recognition site, and exhibit an ability to specifically bind antigen. Such fragments include Fab′, F(ab′)₂, Fv, single chain (ScFv), and mutants thereof, naturally occurring variants, and fusion proteins including the antibody's variable region antigen recognition site and a heterologous protein (e.g., a toxin, an antigen recognition site for a different antigen, an enzyme, a receptor or receptor ligand, etc.).

As used throughout, a subject can be a vertebrate, more specifically a mammal (e.g., a human, horse, cat, dog, cow, pig, sheep, goat, mouse, rabbit, rat, and guinea pig). The term does not denote a particular age or sex. Thus, adult, newborn and pediatric subjects, whether male or female, are intended to be covered. As used herein, patient or subject may be used interchangeably and can refer to a subject with or at risk of developing a disorder. The term patient or subject includes human and veterinary subjects. In any of the methods provided herein, the subject can be a subject diagnosed with cancer, an infection or an autoimmune disease.

As used herein the terms treatment, treat, or treating refers to a method of reducing one or more of the effects of the disorder or one or more symptoms of the disorder, for example, cancer in the subject. Thus in the disclosed methods, treatment can refer to a 10%, 20%, 30%, 40%, 50%, 60%, 70%, 80%, 90%, or 100% reduction in the severity of cancer. For example, a method for treating cancer is considered to be a treatment if there is a 10% reduction in one or more symptoms of the cancer in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disorder or symptoms of the disorder.

As used herein, the term therapeutically effective amount or effective amount refers to an amount of a composition comprising CD4⁺ T_{SCM} cells or cells differentiated therefrom, chemotherapeutic agent, immunotherapeutic agent, etc. described herein, that, when administered to a subject, is effective, alone or in combination with additional agents, to treat a disease or disorder either by one dose or over the course of multiple doses. A suitable dose can depend on a variety of factors including the particular cells or agent used and whether it is used concomitantly with other therapeutic agents. Other factors affecting the dose administered to the subject include, e.g., the type or severity of the disease. For example, a subject having pancreatic cancer may require administration of a different dosage of a composition comprising CD4⁺ T_{SCM} cells or cells differentiated therefrom and/or a chemotherapeutic agent than a subject with breast cancer.

The effective amount of CD4⁺ T_{SCM} cells or cells differentiated therefrom can be determined by one of ordinary skill in the art and includes exemplary amounts for a mammal of about 0.1×10⁵ to about 8×10⁹ cells/kg of body weight.

The effective amount of the compounds (for example, an MEK1/2 inhibitor, chemotherapeutic agent or immunomodulator) described herein or pharmaceutically acceptable salts or prodrugs thereof can be determined by one of ordinary skill in the art and includes exemplary dosage amounts for a mammal of from about 0.5 to about 200 mg/kg of body weight of active compound per day, which can be administered in a single dose or in the form of individual divided doses, such as from 1 to 4 times per day. Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, or about 5 mg/kg of body weight of active compound per day. Other factors that influence dosage can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject also depends upon the judgment of the treating medical practitioner. A therapeutically effective amount is also one in which any toxic or detrimental effects of the composition are outweighed by the therapeutically beneficial effects.

As used herein, administer or administration refers to the act of introducing, injecting or otherwise physically delivering a substance as it exists outside the body (e.g. CD4⁺ T_{SCM} cells, cells differentiated therefrom or any non-cellular therapeutic agent described herein) into a subject, such as by mucosal, intradermal, intravenous, intratumoral, intramuscular, intrarectal, oral, subcutaneous delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof.

Any of the therapeutic agents described herein (e.g., CD4⁺ T_{SCM} cells, cells differentiated therefrom or any other non-cellular therapeutic agent described herein (for example, a chemotherapeutic agent, a vaccine, an immunotherapeutic, etc.) are administered in a number of ways depending on whether local or systemic treatment is desired, and on the area to be treated. The compositions are administered via any of several routes of administration, including orally, parenterally, intramucosally, intravenously, intraperitoneally, intraventricularly, intramuscularly, subcutaneously, intracavity or transdermally. Administration can be achieved by, e.g., topical administration, local infusion, injection, or by means of an implant. The implant can be of a porous, non-porous, or gelatinous material, including membranes, such as sialastic membranes, or fibers. The implant can be configured for sustained or periodic release of the composition to the subject. See, e.g., U.S. Patent Application Publication No. 20080241223; U.S. Pat. Nos. 5,501,856; 4,863,457; and 3,710,795; and European Patent Nos. EP488401 and EP 430539.

In some cases, the CD4⁺ T_{SCM} cells or cells differentiated therefrom can be engineered to comprise a binding or targeting moiety that binds a specific protein, for example, a cancer-specific receptor, such as, for example, a chimeric antigen receptor. See, for example, Eisenberg et al. “Targeting Multiple Tumors Using T-cells Engineered to Express a Natural Cytotoxicity Receptor 2-Based Chimeric Receptor,” Front. Immunol. 8: 1212 (2017); and Figueroa et al. “Chimeric antigen receptor engineering: a right step in the evolution of adoptive cellular immunotherapy,” Int. Rev. Immunol. 34(2): 154-87 (2015).

In some methods, a non-cellular therapeutic agent such as a chemotherapeutic agent, an MEK1/2 inhibitor, an immunotherapeutic agent etc., can be delivered to the subject by way of an implantable device based on, e.g., diffusive, erodible, or convective systems, osmotic pumps, biodegradable implants, electrodiffusion systems, electroosmosis systems, vapor pressure pumps, electrolytic pumps, effervescent pumps, piezoelectric pumps, erosion-based systems, or electromechanical systems. Nanoparticle delivery is also contemplated herein. Effective doses for any of the administration methods described herein can be extrapolated from dose-response curves derived from in vitro or animal model test systems.

The cells and compounds described herein can be formulated as a pharmaceutical composition. In some embodiments, the pharmaceutical composition can further comprise a carrier. The term carrier means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject. Such pharmaceutically acceptable carriers include sterile biocompatible pharmaceutical carriers, including, but not limited to, saline, buffered saline, artificial cerebral spinal fluid, dextrose, and water.

The CD4⁺ T_{SCM} cells or cells differentiated therefrom can be formulated as a pharmaceutical composition for parenteral administration. In some examples, the pharmaceutical composition further comprises a second therapeutic agent, as described herein. The T cells are typically administered in an aqueous solution, by parenteral injection. The formulation may also be in the form of a suspension or emulsion.

Depending on the intended mode of administration, a pharmaceutical composition comprising a non-cellular therapeutic agent described herein, can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the agent described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected agent without causing unacceptable biological effects or interacting in a deleterious manner with the other components of the pharmaceutical composition in which it is contained.

### Compositions

Provided herein are pharmaceutical compositions comprising CD4⁺ T_{SCM} cells, optionally with a second therapeutic agent. The CD4⁺ T_{SCM} cells can be produced by any of the methods provided herein. The second therapeutic agent can be selected from the group consisting of an immunomodulator, a vaccine, a tumor-specific antigen or a pathogen-specific antigen.

In general, pharmaceutical compositions are provided that include effective amounts of CD4⁺ T_{SCM} cells and/or cells differentiated therefrom, and optionally include pharmaceutically acceptable diluents, preservatives, solubilizers, emulsifiers, adjuvants and/or carriers. Such compositions optionally include one or more for the following: diluents, sterile water, buffered saline of various buffer content (e.g., Tris-HCl, acetate, phosphate), pH and ionic strength; and additives such as detergents and solubilizing agents (e.g., TWEEN 20 (polysorbate-20), TWEEN 80 (polysorbate-80)), anti-oxidants (e.g., ascorbic acid, sodium metabisulfite), and preservatives (e.g., Thimersol, benzyl alcohol) and bulking substances (e.g., lactose, mannitol).

Also provided is a kit comprising one or more MEK1/2 inhibitors and CD4⁺ T_{SCM} cells in one or more containers. The kit can further comprise include instructions or labels promoting or describing the use of the cells and compounds of the invention. The kit can also comprise a means for delivery of the cells and/or MEK1/2 inhibitor(s) to the subject.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to one or more molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### Examples

### In Vivo and In Vitro T_{SCM} Generation

TC-1 tumor bearing mice were treated with an MEK inhibitor (MEKi) (10 mg/Kg), for a total of 15 doses, given everyday by oral gavage, starting when tumor reached an average size of 0.05 cm³. Mice were vaccinated twice with cognate antigen at one-week intervals. Two to three days after a second vaccination mice were sacrificed, tumors harvested and processed into a single-cell suspension. The single-cell suspended tumors were labelled with appropriately conjugated fluorophore conjugated antibodies and analyzed by FACS.

### In Vitro Assay

CD4 cells from the splenocytes of wild type (WT) mice were prepared by magnetic purification using Biolegend cell sorting kits, as per the manufacturer's instructions. Purified cells were activated by plating 1×10⁶/ml in cell activation medium that contained rIL-2 (100 IU/ml), anti-CD3 (5 ug/ml), anti-CD28 (2.5 ug/ml) and MEKi (1 uM). Appropriate arms (only cell activation and only IL-2 treatment) were also included for valid comparisons. After 72 hours of incubation at 37° C., at 5% CO₂ in a humidified incubator, cells were picked up and processed for FACS analysis of T_{SCM} induction.

### Cell Lineage Experiments

CD4 cells from WT mice were activated with cell activation medium as noted above (IL-2+anti-CD3+anti-CD28+MEKi). In addition, various cell skewing cytokines were added to the activation medium in the presence of MEKi to test the ability of MEK inhibition on the generation of lineage-specific T_{SCM} cells. The cell skewing conditions used were: Th1 (IL-2 (100 U/mL)+IL-12 (10 ng/mL)+IFN-γ (10 ng/mL)+αIL-4 (10 ug/mL)); Th2 (IL-2 (100 U/mL)+IL-4 (30 ng/mL)+αIL-12 (10 ug/mL)+αIFN-γ (10 ug/mL)); Treg (IL-2 (100 U/mL)+TGFβ (2.5 ng/mL)) and Th17 (TGF β (2.5 ng/mL)+IL-6 (100 ng/mL)) polarizing conditions. Post-activation, different phenotypes of the cells were determined: Th1 (T-bet and IFN-γ), Th2 (GATA-3 and IL-4), Treg (FoxP3 and IL-10), Th17 (ROR-γ and IL-17) and T_{SCM} (Sca1⁺CD62L⁺CD44⁻) by flow cytometry.

### Multipotency

FACS-sorted CD4⁺ T cells from spleens of WT mice were cultured for 72 hours in the presence of IL-2/anti-CD3/anti-CD28 with or without MEK1/2i (Th0 condition). After 72 hours, T_{SCM} (Sca1⁺CD62L⁺CD44⁻) and T_{CM} (CD62L⁺CD44⁺) cells were FACS sorted and further activated with IL-2/anti-CD3/anti-CD28 (Th0) or cell skewing conditions for 24 hours. Th1 (IL-2 (100 U/mL)+IL-12 (10 ng/mL)+IFN-γ (10 ng/mL)+αIL-4 (10 ug/mL)); Th2 (IL-2 (100 U/mL)+IL-4 (30 ng/mL)+αIL-12 (10 ug/mL)+αIFN-γ (10 ug/mL)); Treg (IL-2 (100 U/mL)+TGFβ (2.5 ng/mL)) and Th17 (TGF β (2.5 ng/mL)+IL-6 (100 ng/mL)) polarizing conditions. After incubation cells were stained for respective transcription factors (FoxP3, ROR-γ, Tbet, GATA) and analyzed by flow cytometry.

### Results

### MEK1/2 Inhibition Induces Self-Renewable Multipotent Stem Cell-Like Memory Cells in CD4 Cells.

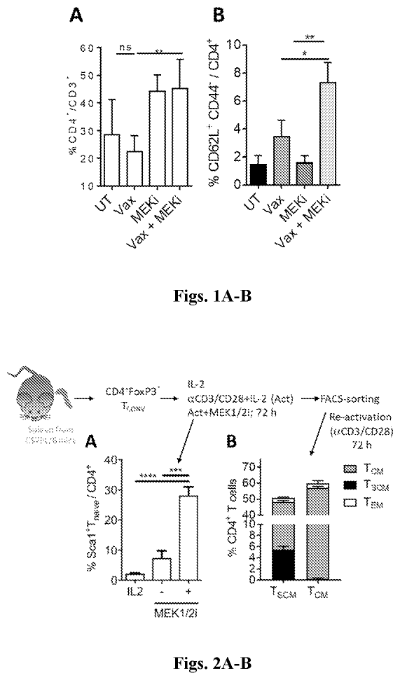

The effects of MEK1/2 inhibition on the frequency of total CD4⁺ T cells and generation of CD4⁺ T_{SCM} cells were determined. It was found that MEK1/2 inhibitor increases the numbers of CD4⁺ T cells in the tumor microenvironment (TME) (FIG. 1A). Importantly, MEK1/2 inhibition significantly enhanced the induction of CD4⁺ T cells with a naïve-like phenotype (CD62L⁺CD44⁻) in the TME (FIG. 1B).

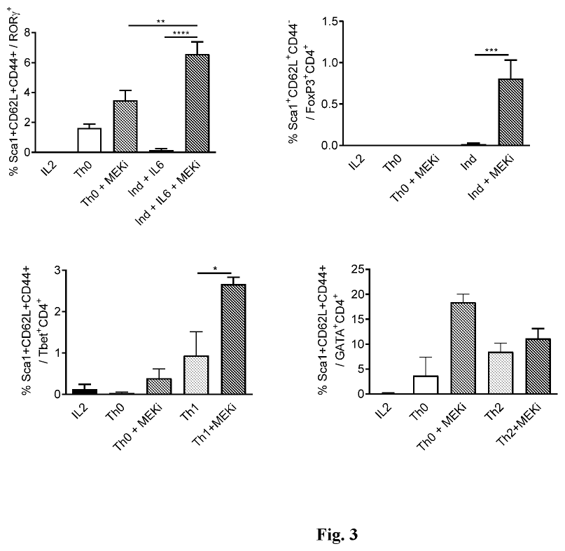

These findings were expanded under in vitro conditions. In mice, T_{SCM} cells are characterized by a naïve-like phenotype (CD62L⁺CD44⁻) with high expression of Sca1 (Rosenblum et al. “Regulatory T cell memory,” Nature reviews. Immunology 16: 90-101, (2016)). Therefore, the number of Sca1⁺ T_{NAIVE} CD4⁺ T cells following MEK1/2 inhibition was determined. T conventional (T_{CONV}) cells activated in the presence of a MEK1/2 inhibitor exhibited significantly enhanced generation of Sca1⁺ T_{NAIVE} CD4⁺ T cells than cells activated without MEK1/2 inhibitor (FIG. 2A). To functionally confirm that Sca1⁺ naïve cells are indeed T_{SCM} cells, their self-renewal capacity and multipotency were determined. When compared to CD4⁺ T_{CM}, MEK1/2 inhibitor-induced T_{SCM} have enhanced self-renewal capacity and multipotency index (FIG. 2B), confirming their stemness.

MEK1/2 Inhibition Generates Lineage-Specific CD4⁺ T_{SCM} Cells.

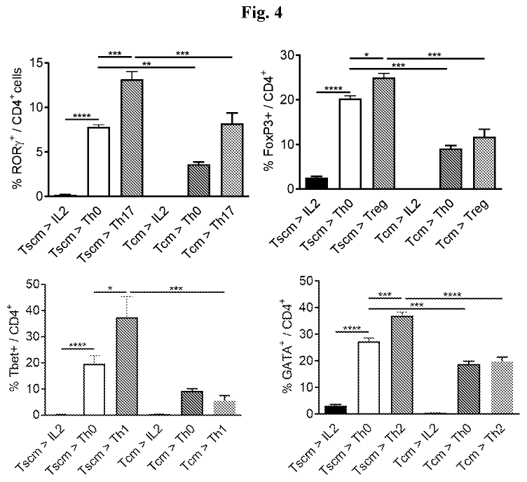

Stem cell memory CD8⁺ and CD4⁺ T cells and B cells are most likely the only mature blood cells other than the hematopoietic stem cells (HSCs) that share features such as self-renewal with HSCs. CD4⁺ naïve T cells constitute a heterogeneous population that harbor diversity in phenotypes, differentiation stages, persistence, functions, and anatomic localizations. These cells represent cellular subsets that are extremely heterogeneous and multifunctional at their very initial stages of differentiation, with the potential to become different types of memory and effector cells. Whether MEK1/2 inhibitor-induced CD4⁺ T_{SCM} cells can be lineage-committed and differentiate into specific CD4⁺ T cell subtypes, if generated under lineage-specific conditions, was determined. (FIG. 3). Indeed, lineage-specific CD4⁺ T_{SCM} cells could be generated by inhibiting MEK1/2 in CD4⁺ T cells during their activation in specific cell skewing conditions (FIG. 3A-D).

### MEK1/2 Inhibition Generates Multipotent T_{SCM} Cells.

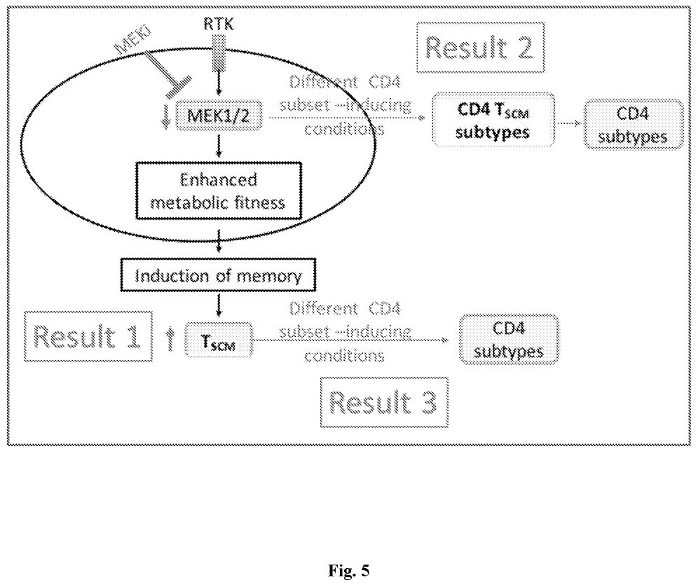

T_{SCM} cells are naïve-like cells that can be skewed into various cell-types depending upon the signals they receive (Luckey et al. “Memory T and memory B cells share a transcriptional program of self-renewal with long-term hematopoietic stem cells,” Proc Natl Acad Sci USA 103, 3304-3309, (2006); Caccamo et al. “Atypical Human Effector/Memory CD4(+) T Cells With a Naive-Like Phenotype,” Front Immunol 9: 2832 (2018)). CD4⁺ T cells comprise a diverse population in which each subtype of helper T cell has different functions (Kennedy et al. “Multiple roles for CD4⁺ T cells in anti-tumor immune responses,” Immunol Rev 222:129-144, (2008)). For example, Treg cells can be beneficial in disease settings where reduction of inflammation is required, such as autoimmune disorders. Moreover, within pro-inflammatory cell types (Th1, Th2, Th17), the anti-tumor efficacy may be significantly different. Hence, the ability to generate various cell types is of strong value in addressing various disease conditions. Therefore, the multipotency of T_{SCM} cells generated after MEK1/2 inhibition of CD4 cells was checked by analyzing if they can be polarized into different CD4 subtypes: Th1, Th2, Treg and Th17. For this, FACS-sorted CD4⁺ T_{SCM} cells were further activated with or without cell lineage-specific conditions. With the exception of FoxP3, T_{SCM} cells were capable of expressing various lineage-specific transcription factors even without cell skewing conditions. However, importantly, the expression of respective transcription factors was significantly increased in respective cell skewing conditions. These results show that T_{SCM} cells and not T_{CM} cells have the multipotent ability to generate other CD4 T cell phenotypes (FIG. 4).

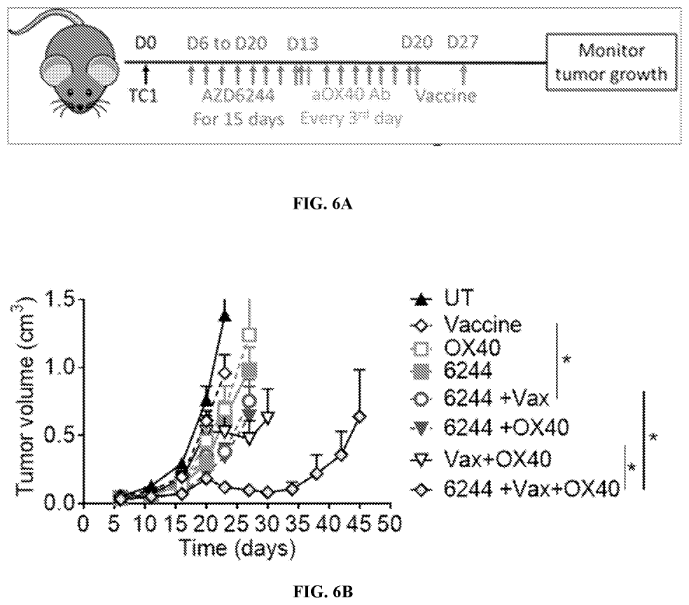

As shown in FIG. 5, the data provided herein show that MEK1/2 inhibition induces CD4⁺ T_{SCM} cells that have self-renewal capacity with potent recall response. It was also found that cell lineage-specific CD4⁺ T_{SCM} cells can be generated by providing specific cell skewing conditions. Furthermore, it was found that the CD4⁺ T_{SCM} generated after MEK1/2 inhibition are multipotent i.e. these T_{SCM} cells are able to generate other CD4⁺ T cell phenotypes after their TCR-mediated activation.

### MEK1/2 Inhibition Enhances the Anti-Tumor Efficacy of an Anti-OX40 Antibody

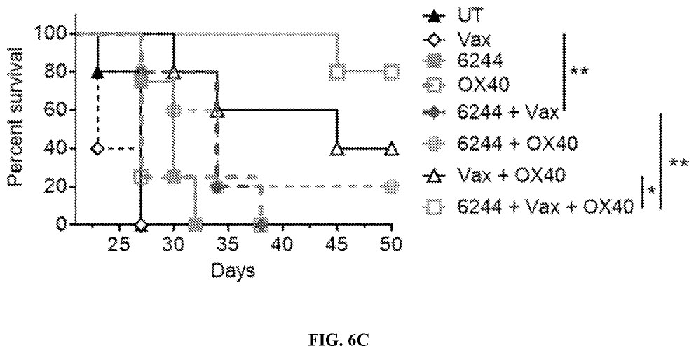

C57BL/6 mice were inoculated with 70,000 TC1 cells/mouse in the right flank at Day 0 (DO). By D6, when the tumor reached an average volume of 0.05 cm³, MEKi treatment (ASD6244, selumitinib) (10 mg/Kg) was started by oral gavage. This treatment was administered for 15 days (D6-D20). On Day 8-9, when the tumor reached an average size of 0.07-0.08 cm³, mice were vaccinated using a tumor specific E7 peptide (100 μg/mouse; subcutaneous) either alone or in combination with an anti-OX40 antibody (Clone: OX86; 1 mg/kg, intraperitoneal). E7 peptide was administered three times, at an interval of one week between various immunizations. Anti-OX40 antibody was administered every third day for the duration of the experiment. A schematic showing the timeline for this experiment is shown in FIG. 6A. Mice were sacrificed when the tumor reached a volume of 1.5 cm³. As shown in FIG. 6B, in vaccinated animals, MEKi (6244) in combination with an anti-OX40 antibody, significantly reduced the tumor growth. There was also a simultaneous significant increase in mice survival, as shown in FIG. 6C.

1. A method for producing stem cell-like memory T (T_{SCM}) cells comprising contacting CD4⁺ T cells in vitro or ex vivo with an effective amount of an MEK1/2 inhibitor to produce CD4⁺ T_{SCM} cells.

2. The method of claim 1, wherein the method comprises concurrently contacting the CD4⁺ T cells with an effective amount of an MEK1/2 inhibitor and cell-lineage specific inducing conditions to produce one or more types of cell-lineage specific CD4⁺ T_{SCM} cells.

3. The method of claim 2, wherein the cell-lineage specific CD4⁺ T_{SCM} cells are selected from the group consisting of Treg, Th1, Th2 and Th17 cells.

4. The method of claim 1, wherein the CD4⁺ T_{SCM} cells are multipotent and wherein the method further comprises contacting the multipotent CD4⁺ T_{SCM} cells with cell-lineage specific inducing conditions to differentiate the multipotent CD4⁺ T_{SCM} cells into one or more types of cells of CD4⁺ specific T cell-lineage.

5. The method of claim 4, wherein the one or more types of cells that are of CD4⁺ specific T cell-lineage are selected from the group consisting of Treg, Th1, Th2 and Th17 cells.

6. The method of claim 5, wherein the type of cell of CD4⁺ specific T cell lineage is a Treg cell and wherein differentiating the T_{SCM} cells into Treg cells comprises contacting the T_{SCM} cells with IL-2 and TGFβ.

7. The method of claim 5, wherein the type of cell of CD4⁺ specific T cell lineage is a Th1 cell and wherein differentiating the T_{SCM} cells into Th1 cells comprises contacting the T_{SCM} cells with IL-2, IL-12, IFN-γ and αIL-4.

8. The method of claim 5, wherein the type of cell of CD4⁺ specific T cell lineage is a Th2 cell and wherein differentiating the T_{SCM} cells into Th2 cells comprises contacting the T_{SCM} cells with IL-2, IL-4, αIL-12 and αIFN-γ.

9. The method of claim 5, wherein the type of cells that are of CD4⁺ specific T cell lineage is a Th17 cell, and wherein differentiating the T_{SCM} cells into Th17 cells comprises contacting the T_{SCM} cells with TGFβ and IL-6.

10. The method of claim 1, further comprising expanding the CD4⁺ T_{SCM} cells in culture.

11. The method claim 1, wherein the T_{SCM} cells have a CD62L⁺CD44⁻ naïve-like phenotype.

12. The method of claim 1, wherein the CD4⁺ T_{SCM} cells have an increased level of Sca1 as compared to untreated CD4⁺ T cells.

13. The method of claim 1, wherein the MEK1/2 inhibitor is Selumetinib.

14. The method of claim 1, wherein the CD4⁺ T cells are genetically engineered CD4⁺ T cells.

15. The method of claim 14, wherein the CD4⁺ T cells are genetically engineered to express a chimeric antigen receptor.

16. A method for treating an infection or cancer in a subject comprising:
a) contacting CD4⁺ T cells ex vivo with an effective amount of an MEK1/2 inhibitor to produce T_{SCM} cells; and
b) administering the T_{SCM} cells to a subject with an infection or cancer.

17. The method of claim 16, wherein the T_{SCM} cells are expanded prior to administration to the subject.

18. The method of claim 16, further comprising differentiating the T_{SCM} cells into Th1, Th2 or Th17 cells prior to administration to the subject.

19. The method of claim 16, wherein the CD4⁺ T cells are genetically engineered CD4⁺ T cells.

20. The method of claim 19, wherein the CD4⁺ T cells are genetically engineered to express a chimeric antigen receptor.

21. The method of claim 16, wherein the CD4⁺ T cells are autologous CD4⁺ T cells.

22. The method of claim 16, wherein the CD4⁺ T cells are homologous CD4⁺ T cells.

23. The method of claim 16, further comprising administering an effective amount of a second therapeutic agent to the subject.

24. The method of claim 23, wherein the second therapeutic agent is selected from the group consisting of an immunomodulatory agent, a vaccine, a tumor antigen or a pathogen antigen.

25. The method of claim 24, wherein the immunomodulator is an antibody or an antigen binding fragment thereof that binds to PD1, PDL1, OX40, CTLA-4, TIM-3, TIGIT, VISTA, BTLA, LAG-3, CD27, KIR, A2AR or GITR.

26. The method of claim 24, wherein the immunomodulator is an immunosuppressant.

27. The method of claim 24, wherein the immunomodulator is an immunostimulant.

28. A method for treating an autoimmune disorder in a subject comprising:
a) contacting CD4⁺ T cells ex vivo with an effective amount of an MEK1/2 inhibitor to produce T_{SCM} cells; and
b) administering the T_{SCM} cells to the subject with an autoimmune disorder.

29. The method of claim 28, wherein the T_{SCM} cells are expanded prior to administration to the subject.

30. The method of claim 28, further comprising differentiating the T_{SCM} cells into Treg cells prior to administration to the subject.

31. The method of claim 28, wherein the CD4⁺ T cells are genetically engineered CD4⁺ T cells.

32. The method of claim 30, wherein the CD4⁺ T cells are genetically engineered to express a chimeric antigen receptor.

33. The method of claim 28, wherein the CD4⁺ T cells are autologous CD4⁺ T cells.

34. The method of claim 28, wherein the CD4⁺ T cells are homologous CD4⁺ T cells.

35. The method of claim 28, further comprising administering an effective amount of an immunosuppressant to the subject.

36. A pharmaceutical composition comprising:
a) a cell produced by the method of claim 1; and
b) a second therapeutic agent.

37. The method of claim 36, wherein the second therapeutic agent is selected from the group consisting of an immunomodulator, a vaccine, a tumor-specific antigen or a pathogen-specific antigen.

38. The pharmaceutical composition of 37, wherein the vaccine comprises a tumor specific antigen.

39. A method of treating cancer in a subject comprising administering to the subject the composition of claim 36.

40. A method of treating an infection or an autoimmune disorder comprising administering to the subject the composition of claim 36.
